(19) United States
(12) Patent Application Publication
(10) Pub. No.: US 2002/0106648 A1
(43) Pub. Date: Aug. 8, 2002

(54) Highly multiplexed reporter carrier systems

(76) Inventors: Paul M. Lizardi, Wallingford, CT (US); Brian T. Chait, New York, NY (US); Darin R. Latimer, East Haven, CT (US)

Correspondence Address: Robert A. Hodges, Ph.D., NEEDLE & ROSENBERG, P.C., The Candler Building, Suite 1200, 127 Peachtree Street, N.E., Atlanta, GA, 30303-1811, US

(21) Appl. No.: 09/850,539

(22) Filed: May 7, 2001

Related U.S. Application Data
(60) Provisional application No. 60/201,963, filed on May 5, 2000.
(60) Provisional application No. 60/224,939, filed on Aug. 11, 2000.

(51) Int. Cl. C12Q 1/68

(52) U.S. Cl. 435/6

(57) ABSTRACT

Disclosed are a composition and a method for a multiplexing-optimized reporter system. The system is designed for the simultaneous detection of dozens or even hundreds of analytes. The analytes may be present on the surface of cells in suspension, on the surface of cytology smears, on the surface of histological sections, on the surface of DNA microarrays, on the surface of protein microarrays, on the surface of beads, or any other situation where complex samples need to be studied. The disclosed composition accomplishes this detection by associating specific binding molecules—which interact with desired targets—with numerous tag molecules in a carrier. The numerous tag molecules can be detected and effectively amplify the signal generated from targets.

### CROSS-REFERENCE TO RELATED APPLICATIONS

This application claims benefit of U.S. Provisional Application No. 60/201,963, filed May 5, 2000, and U.S. Provisional Application 60/224,939, filed Aug. 11, 2000. Application Ser. No. 60/201,963, filed May 5, 2000, and Application No. 60/224,939, filed Aug. 11, 2000, are hereby incorporated herein by reference.

The present invention is generally in the field of detection of molecules, and specifically in the field of detection of multiple different molecules in a single assay.

It is an object of the present invention to provide a composition that permits the indirect detection of a large number of different analytes in a single sample or group of samples.

It is another object of the present invention to provide a composition that permits the indirect detection of a large number of different proteins in a single sample or group of samples.

### BACKGROUND OF THE INVENTION

The analysis of proteins in histological sections and other cytological preparations is routinely performed using the techniques of histochemistry, immunohistochemistry, or immunofluorescence. By performing immunofluorescence with antibodies labeled with different colors, it has been possible to detect simultaneously 2, 3, or even 4 different antigens present in cellular material. In the future, time-resolved fluorescence may permit the extension of immunofluorescence methods to the detection of 6 to 12 different antibodies simultaneously. Likewise, RNA detection by fluorescence in situ hybridization permits the detection of 2 to 4 different RNAs in cellular material, and it may also be extended to permit the detection of 6 to 12 different RNAs by time-resolved fluorescence.

There is a need for a sensitive method that will permit the cytological detection of larger numbers of proteins or RNAs simultaneously. Theoretically, the simultaneous measurement of the concentration of 20 to 50 different protein (or RNA) species should be highly informative as to the specific status of dynamic cellular processes in normal development, in stages of disease, in response to drug treatment or gene therapy, or as a result of environmental exposure or other deliberate or inadvertent interventions.

The study of cells by measuring the identity and concentration of a relatively large number of proteins simultaneously (referred to as proteomics) is currently a very time-consuming task. Two-dimensional (2D) gel electrophoresis is the most powerful tool for studying the expression of multiple proteins, but this technique is not readily adaptable to in-situ cell analysis. Typically, many thousands of cells are required to perform a single 2D gel analysis. In order to identify different protein expression profiles in heterogeneous tissue samples, one would need the capability to analyze the proteins expressed in a small number of cells. This capability is most relevant in the analysis of histological or cytological specimens that may harbor dysplastic or pre-malignant cells. Such cells, which may precede the development of cancer, need to be identified when present as small foci of 10 to 50 cells, before they have a chance to give rise to tumors. Unfortunately, the amount of protein obtained from 10 to 50 cells is insufficient for 2D gel analysis, and is problematic even with the use of radioisotopes to label the protein.

Mass spectroscopy is another powerful technique for protein analysis. However, the direct analysis of proteins present in samples containing small numbers of cells is not possible with prior mass spectroscopy technology, due to insufficient sensitivity. A minimum of 10,000 cells is required for mass spectroscopic analysis of tissue samples using prior technology.

Current methods for the analysis of microarray hybridization experiments rely on the use of a two-color signal readout system. For example, Schena M, Shalon D, Davis R W, Brown P O (1995) Quantitative monitoring of gene expression patterns with a complementary DNA microarray. Science 270:467-70, describe an experiment where cDNA prepared from one tissue is labeled with the dye cy3, while cDNA from another tissue is labeled with the dye cyS. After the labeling reactions are performed, the two labeled DNAs are mixed, and hybridized by contacting with the surface of a glass slide containing a cDNA microarray on its surface. At the end of the hybridization reaction, the microarray surface is washed to remove unhybridized material, and the glass slide is scanned in a confocal scanning instrument designed to record separately the cy3 and the cy5 fluorescence intensity, which is saved as two different computer files. Computer software is then used to calculate the fluorescence ratio of cy3 to cy5 at each of the specific dot-addresses on the DNA microarray. This experimental design works very well for performing comparisons of mRNA expression ratios between two samples.

Gygi SP, Rist B, Gerber S A, Turecek F, Gelb M H, Aebersold R (1999) Quantitative analysis of complex protein mixtures using isotope-coded affinity tags. Nature Biotechnology 17:994-999, have described an approach for the accurate quantification and concurrent sequence identification of the individual proteins within complex mixtures of biological origin. The method is based on a class of new chemical reagents termed isotope-coded affinity tags (ICATs), and tandem mass spectrometry. These authors extracted proteins from two different experimental states of an organism, and labeled each of the two preparations of total protein with two different thiol-reactive ICAT tags of different mass. The two labeled protein preparations were mixed, separated by liquid chromatography, and detected on line by mass spectrometry. For each individual protein peak, mass spectrometry permitted protein identification, as well as measurement of the ratio of the amounts of the two proteins.

### BRIEF SUMMARY OF THE INVENTION

Disclosed are compositions and a method for a multiplexing-optimized reporter system. The system is designed for the simultaneous detection of dozens or even hundreds of analytes. The analytes may be present on the surface of cells in suspension, on the surface of cytology smears, on the surface of histological sections, on the surface of DNA microarrays, on the surface of protein microarrays, on the surface of beads, or any other situation where complex samples need to be studied. The disclosed composition accomplishes this detection by associating specific binding molecules—which interact with desired targets—with numerous tag molecules in a carrier. The numerous tag molecules can be detected and effectively amplify the signal generated from targets.

### DETAILED DESCRIPTION OF THE INVENTION

Disclosed are a composition and a method for a multiplexing-optimized reporter system. The system is designed for the simultaneous detection of dozens or even hundreds of analytes. The analytes may be present on the surface of cells in suspension, on the surface of cytology smears, on the surface of histological sections, on the surface of DNA microarrays, on the surface of protein microarrays, on the surface of beads, or any other situation where complex samples need to be studied.

Methods currently in use for the detection of protein, DNA, or RNA in biological material are limited to the use of just a few analytes as targets. There is a need for methods that enable the simultaneous detection of a large number of analytes. Certain microarray methods solve this multiplexing requirement by distributing samples so they are physically separated at different addresses on a surface. However, there are certain types of analysis where the analytes in a sample of interest are all present in a single address or location, and can not be separated physically. For example, one may desire to detect 40 different proteins simultaneously, with fairly accurate cellular localization, on the surface of a tissue section (fairly accurate means that the proteins are detected in the vicinity of a group of 20 cells or less. This type of information may be useful, for example, to decide if a certain group of cells has undergone neoplastic transformation.

Different embodiments of the present compositions and method allow the detection of protein, RNA, DNA, carbohydrate, or any other analyte of interest, based on the use of specific recognition moieties for each of these analytes. For example, the preferred recognition moiety for a protein analyte is an antibody specific for an epitope present in that protein, while the preferred recognition moiety for a nucleic acid analyte is a complementary nucleic acid probe.

The disclosed compositions, referred to herein as reporter carriers, are based on the use of carriers loaded with a plurality of arbitrary molecular tags that have been optimized to facilitate a subsequent detection. The carriers are linked, preferably by covalent coupling, to specific recognition molecules. The carriers, by virtue of the directly or indirectly linked recognition molecules, may be used as reporters in bioassays. The molecular tags preferably are optimized by their chemical composition, so that they may be efficiently separated by standard methods, such as mass spectrometry, electrophoresis, or liquid chromatography. Tags to be separated by mass spectrometry will differ in molecular weight, preferably by well resolved mass differences that allow for reliable separation. For separation by electrophoresis, the carriers may be loaded with DNA oligonucleotides in the range of 20 to 300 nucleotides, where differences of 2 or more bases are sufficient for good separation.

Reporter Carriers

Reporter carriers are associations of one or more specific binding molecules, a carrier, and a plurality of decoding tags. Reporter carriers are used in the disclosed method to associate a large number of decoding tags with a target molecule. The carrier can be any molecule or structure that facilitates association of many decoding tags with a specific binding molecule. Examples include liposomes, microparticles, nanoparticles, virons, phagmids, and branched polymer structures. The are three preferred types of reporter carriers: liposome reporter carriers, dendrimer reporter carriers, and microbead reporter carriers.

Liposome Reporter Carriers

Liposomes are artificial structures primarily composed of phospholipid bilayers. Cholesterol and fatty acids may also be included in the bilayer construction. Liposomes may be loaded with fluorescent tags, and coated on the outer surface with specific recognition molecules (Truneh, A., Machy, P. and Horan, P. K., 1987, Antibody-bearing liposomes as multicolor immunofluorescent markers for flow cytometry and imaging. J. Immunol. Methods 100:59-71). However, the use of fluorescent liposomes in bioassays has been limited by the constraints of detection methods for fluorescent tags. Fluorescence-activated cell sorters typically have two or three different excitation-emission wavelengths, and microscopes typically have three or four excitation-emission filters. In the disclosed liposome reporter carriers, liposomes serve as carriers for arbitrary decoding tags. By combining liposome reporter carriers, loaded with arbitrary tags, with methods capable of separating a very large multiplicity of tags, it becomes possible to perform highly multiplexed assays.

Liposomes, preferably unilamellar vesicles, are made using established procedures that result in the loading of the interior compartment with a very large number (several thousand) of decoding tag molecules, where the chemical nature of these molecules is well suited for detection by a preselected detection method. Preferred combinations of detection methods and corresponding arbitrary tags are: mass spectrometry for detection of oligopeptide tags, electrophoresis for detection of DNA oligonucleotide tags, and liquid chromatography for detection of DNA tags or oligopeptide tags. The molecular tags are designed to serve as decoding entities for the assay. Thus, one specific type of decoding tag preferably is used for each specific type of liposome reporter carrier.

Each specific type of liposome reporter carrier is associated with a specific binding molecule. The association may be direct or indirect. An example of a direct association is a liposome containing covalently bound antibodies on the surface of the phospholipid bilayer. An example of indirect association is a liposome containing covalently bound nucleic acid of arbitrary sequence on its surface. These oligonucleotides are designed to recognize, by base complementarity, specific reporter molecules. The reporter molecule may comprise an antibody-DNA covalent complex, whereby the DNA portion of this complex can hybridize specifically with the complementary sequence on a liposome reporter carrier. In this fashion, the liposome reporter carrier becomes a generic reagent, which may be associated indirectly with any desired binding molecule.

Dendrimer Reporter Carriers

The synthesis of dendrimers that may be used as polylabeled DNA probes has been described (Schchepinov, M. S., Udalova, I. A., Bridgman, A. J., Southern, E. M., 1997, Nucleic Acids Res. 25:4447-4454). Dendrimers may be associated with complementary DNA or PNA (peptide nucleic acid) molecules by hybridization. These hybridized molecules serve as tags for detection. The tags may be optimized by their chemical composition, so that they may be efficiently separated by standard methods, such as mass spectrometry, electrophoresis, of liquid chromatography. Tags to be separated by mass spectrometry will differ in molecular weight, preferably by well resolved mass differences that allow for reliable separation. Tag to be separated by mass spectrometry are preferably PNA (Griffin, T. J., W. Tang, and L. M. Smith, Genetic analysis by peptide nucleic acid affinity MALDI-TOF mass spectrometry. Nat Biotechnol, 1997. 15(12): p. 1368-72) or DNA designed to be resistant to fragmentation (Ono, T., M. Scalf, and L. M. Smith, 2′-Fluoro modified nucleic acids: polymerase-directed synthesis, properties and stability to analysis by matrix-assisted laser desorption/ionization mass spectrometry. Nucleic Acids Res, 1997. 25(22): p. 4581-8).

For separation by electrophoresis, the dendrimer carriers may be loaded with DNA oligonucleotides in the range of 20 to 300 nucleotides, where differences of 2 or more bases are sufficient for good separation.

Specific Binding Molecules

A specific binding molecule is a molecule that interacts specifically with a particular molecule or moiety. The molecule or moiety that interacts specifically with a specific binding molecule is referred to herein as a target molecule. It is to be understood that the term target molecule refers to both separate molecules and to portions of such molecules, such as an epitope of a protein, that interacts specifically with a specific binding molecule. Antibodies, either member of a receptor/ligand pair, synthetic polyamides (Dervan, P. B. and R. W. Burli, Sequence-specific DNA recognition by polyamides. Curr Opin Chem Biol, 1999. 3(6): p. 688-93. Wemmer, D. E. and P. B. Dervan, Targeting the minor groove of DNA. Curr Opin Struct Biol, 1997. 7(3): p. 355-61.), and other molecules with specific binding affinities are examples of specific binding molecules, useful as the affinity portion of a reporter binding molecule.

A specific binding molecule that interacts specifically with a particular target molecule is said to be specific for that target molecule. For example, where the specific binding molecule is an antibody that binds to a particular antigen, the specific binding molecule is said to be specific for that antigen. The antigen is the target molecule. The reporter carrier containing the specific binding molecule can also be referred to as being specific for a particular target molecule. Specific binding molecules preferably are antibodies, ligands, binding proteins, receptor proteins, haptens, aptamers, carbohydrates, synthetic polyamides, or oligonucleotides. Preferred binding proteins are DNA binding proteins. Preferred DNA binding proteins are zinc finger motifs, leucine zipper motifs, helix-turn-helix motifs. These motifs can be combined in the same specific binding molecule.

Antibodies useful as specific binding molecules, can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems.

Properties of zinc fingers, zinc finger motifs, and their interactions, are described by Nardelli, J., T. Gibson, and P. Charnay, Zinc finger-DNA recognition: analysis of base specificity by site-directed mutagenesis. Nucleic Acids Res, 1992. 20(16): p. 4137-44, Jamieson, A. C., S. H. Kim, and J. A. Wells, In vitro selection of zinc fingers with altered DNA-binding specificity. Biochemistry, 1994. 33(19): p. 5689-95, Chandrasegaran, S. and J. Smith, Chimeric restriction enzymes: what is next? Biol Chem, 1999. 380(7-8): p. 841-8, and Smith, J., J. M. Berg, and S. Chandrasegaran, A detailed study of the substrate specificity of a chimeric restriction enzyme. Nucleic Acids Res, 1999. 27(2): p. 674-81.

One form of specific binding molecule is an oligonucleotide or oligonucleotide derivative. Such specific binding molecules are designed for and used to detect specific nucleic acid sequences. Thus, the target molecule for oligonucleotide specific binding molecules are nucleic acid sequences. The target molecule can be a nucleotide sequence within a larger nucleic acid molecule. An oligonucleotide specific binding molecule can be any length that supports specific and stable hybridization between the reporter binding probe and the target molecule. For this purpose, a length of 10 to 40 nucleotides is preferred, with an oligonucleotide specific binding molecule 16 to 25 nucleotides long being most preferred. It is preferred that the oligonucleotide specific binding molecule is peptide nucleic acid. Peptide nucleic acid forms a stable hybrid with DNA. This allows a peptide nucleic acid specific binding molecule to remain firmly adhered to the target sequence.

This useful effect can also be obtained with oligonucleotide specific binding molecules by making use of the triple helix chemical bonding technology described by Gasparro et al., Nucleic Acids Res. 1994 22(14):2845-2852 (1994). Briefly, the oligonucleotide specific binding molecule is designed to form a triple helix when hybridized to a target sequence. This is accomplished generally as known, preferably by selecting either a primarily homopurine or primarily homopyrimidine target sequence. The matching oligonucleotide sequence which constitutes the specific binding molecule will be complementary to the selected target sequence and thus be primarily homopyrimidine or primarily homopurine, respectively. The specific binding molecule (corresponding to the triple helix probe described by Gasparro et al.) contains a chemically linked psoralen derivative. Upon hybridization of the reporter binding probe to a target sequence, a triple helix forms. By exposing the triple helix to low wavelength ultraviolet radiation, the psoralen derivative mediates cross-linking of the probe to the target sequence.

Decoding Tags

Decoding tags are any molecule or moiety that can be associated with a carrier and which can be specifically detected. In particular, different decoding tags should be distinguishable upon detection. Decoding tags preferably are oligonucleotides, carbohydrates, synthetic polyamides, peptide nucleic acids, antibodies, ligands, proteins, peptides, haptens, zinc fingers, aptamers, or mass labels.

Preferred decoding tags are molecules capable of hybridizing specifically to an oligonucleotide reporter tag. Most preferred are peptide nucleic acid decoding tags. Oligonucleotide or peptide nucleic acid decoding tags can have any arbitrary sequence. The only requirement is hybridization to reporter tags. The decoding tags can each be any length that supports specific and stable hybridization between the reporter tags and the decoding tags. For this purpose, a length of 10 to 35 nucleotides is preferred, with a reporter tag 15 to 20 nucleotides long being most preferred.

Decoding tags can be detected using any suitable detection technique. Many molecular detection techniques are known and can be used in the disclosed method. For example, decoding tags can be detected by nuclear magnetic resonance, electron paramagnetic resonance, surface enhanced raman scattering, surface plasmon resonance, fluorescence, phosphorescence, chemiluminescence, resonance raman, microwave, mass spectrometry, or any combination of these. Decoding tags can be separated and/or detected by mass spectrometry, electrophoresis, or chromatography. Decoding tags can be distinguished temporally via different fluorescent, phosphorescent, or chemiluminescent emission lifetimes. The composition and characteristics of decoding tags should be matched with the chosen detection method.

Decoding tags preferably are capable of being released by matrix- assisted laser desorption-ionization (MALDI) in order to be separated and identified (decoded) by time-of-flight (TOF) mass spectroscopy, or of being subjected to electrophoresis. A decoding tag may be any oligomeric molecule that can hybridize to a reporter tag. For example, a decoding tag can be a DNA oligonucleotide, an RNA oligonucleotide, or a peptide nucleic acid (PNA) molecule. Preferred decoding tags are PNA molecules.

For MALDI-TOF detection, the decoding tags preferably are peptide nucleic acids, where each decoding tag has a different mass to allow separation and separate detection in mass spectroscopy. For this purpose, it is preferable that each decoding tag have a similar number of nucleotide bases complementary to the reporter tag. This allows for more consistent hybridization characteristics while allowing the mass to vary. It is also preferable to use combination of base composition and number of mass tags (e.g. the number of 8-amino-3,6-dioxaoctanoic monomers attached to the PNA (Griffin, T. J., W. Tang, and L. M. Smith, Genetic analysis by peptide nucleic acid affinity MALDI-TOF mass spectrometry. Nat Biotechnol, 1997. 15(12): p. 1368-72.)) to optimize the mass spectra for the set of decoding tags in a multiple tag analysis.

For capillary electrophoresis detection, the decoding tags preferably are fluorescently-labeled oligonucleotides, where each decoding tag has a different combination of length and fluorescent label. For this purpose, it is preferable that each decoding tag has the same number of nucleotides complementary to the reporter tag. It is also preferable that each decoding tag has a different number of nucleotides not complementary to the reporter tag. This allows for more consistent hybridization characteristics while allowing separation of the different decoding tags during electrophoresis.

Isobaric Decoding Tags

Preferred decoding tags are isobaric decoding tags. Isobaric decoding tags have two key features. First, the isobaric decoding tags are used in sets where all the isobaric decoding tags in the set have similar properties (such as similar mass-to-charge ratios). The similar properties allow the isobaric decoding tags to be separated from other molecules lacking one or more of the properties. Second, all the isobaric decoding tags in a set can be fragmented, decomposed, reacted, derivatized, or otherwise modified to distinguish the different isobaric decoding tags in the set. Preferably, the isobaric decoding tags are fragmented to yield fragments of similar charge but different mass.

Differential distribution of mass in the fragments of the isobaric decoding tags can be accomplished in a number of ways. For example, isobaric decoding tags of the same nominal structure (for example, peptides having the same amino acid sequence) can be made with different distributions of heavy isotopes, such as deuterium; isobaric decoding tags of the same nominal structure can be made with different distributions of modifications, such as methylation, phosphorylation, sulfation, and use of seleno-methionine for methionine; isobaric decoding tags of the same nominal composition (for example, made up of the same amino acids) can be made with different ordering of the subunits or components of the signal; and isobaric decoding tags having the same nominal composition can be made with a labile or scissile bond at a different location in the signal. Each of these modes can be combined with each other and/or one or more of the other modes to produce differential distribution of mass in the fragments of the isobaric decoding tags. Different sets of isobaric decoding tags can be used together. In this case, it is preferred that the shared property within each different set be different from the shared property of the other sets. For example, the isobaric decoding tags in each set of isobaric decoding tags would have a different mass-to-charge ratio than the isobaric decoding tags in the other sets.

The isobaric decoding tags are preferably detected using mass spectrometry which allows sensitive distinctions between molecules based on their mass-to-charge ratios. A set of isobaric decoding tags are preferred for multiplex detection of many analytes using the disclosed reporter carriers since the isobaric decoding tag fragments can be designed to have a large range of masses, with each mass individually distinguishable upon detection.

A preferred mode of detecting isobaric decoding tags involves filtering of isobaric decoding tags from other molecules based on mass-to-charge ratio, fragmentation of the isobaric decoding tags to produce fragments having different masses, and detection of the different fragments based on their mass-to-charge ratios. The detection is best carried out using a tandem mass spectrometer where the isobaric decoding tags are passed through a filtering quadrupole, the isobaric decoding tags are fragmented in a collision cell, and the fragments are distinguished and detected in a time-of-flight (TOF) stage. In such an instrument the sample is ionized in the source (for example, in a MALDI ion source) to produce charged ions. It is preferred that the ionization conditions are such that primarily a singly charged parent ion is produced. A first quadrupole, Q0, is operated in radio frequency (RF) mode only and acts as an ion guide for all charged particles. The second quadrupole, Q1, is operated in RF+DC mode to pass only a narrow range of mass-to-charge ratios (that includes the mass-to-charge ratio of the isobaric decoding tags). This quadrupole selects the mass-to-charge ratio of interest. Quadrupole Q2, surrounded by a collision cell, is operated in RF only mode and acts as ion guide. The collision cell surrounding Q2 can be filled to appropriate pressure with a gas to fracture the input ions by collisionally induced dissociation. The collision gas preferably is chemically inert, but reactive gases can also be used. Preferred isobaric decoding tags contain scissile bonds, labile bonds, or combinations, such that these bonds will be preferentially fractured in the Q2 collision cell.

Preferred isobaric decoding tags are peptides, oligonucleotides, peptide nucleic acids, carbohydrates, polymers, and combinations of these. Most preferred are peptides. Preferred isobaric decoding tags can be fragmented in tandem mass spectrometry. Peptide-DNA conjugates (Olejnik et al., Nucleic Acids Res., 27(23):4626-31 (1999)), synthesis of PNA-DNA constructs, and special nucleotides such as the photocleavable universal nucleotides of WO 00/04036 can be used as isobaric decoding tags in the disclosed method. Useful photocleavable linkages are also described by Marriott and Ottl, Synthesis and applications of heterobifunctional photocleavable cross-linking reagents, Methods Enzymol. 291:155-75 (1998).

Use of Reporter Carriers

The disclosed reporter carriers are preferably used in a method of detecting multiple analytes in a sample in a single assay. The method is based on encoding target molecules with signals followed by decoding of the encoded signal. This encoding/decoding uncouples the detection of a target molecule from the chemical and physical properties of the target molecule. In basic form, the disclosed method involves association of one or more reporter carriers with one or more target samples—where the reporter carrier includes associated decoding tags—and detection of the decoding tags. The reporter carriers associate with target molecules in the target sample(s). Generally, the reporter carriers correspond to one or more target molecules, and the decoding tags correspond to one or more reporter carriers. Thus, detection of particular decoding tags indicates the presence of the corresponding reporter carriers. In turn, the presence of particular reporter carriers indicates the presence of the corresponding target molecules.

This indirect detection uncouples the detection of target molecules from the chemical and physical properties of the target molecules by interposing decoding tags that essentially can have any arbitrary chemical and physical properties. In particular, decoding tags can have specific properties useful for detection, and decoding tags within an assay can have highly ordered or structured relationships with each other. It is the (freely chosen) properties of the decoding tags, rather than the (take them as they are) properties of the target molecules that matters at the point of detection.

The decoding tags have the additional advantage of being uncoupled from the target molecule-specific aspects of the reporter carriers. Unlike detection methods where a labeled molecule is bound to an analyte followed by detection of the label, the disclosed method is not limited by the chemical and physical properties of the labeled molecule. This allows more convenient detection, more sensitive detection, and more highly multiplexed detection schemes.

### Illustrations

Illustration 1:

The following illustrates use of examples of the disclosed liposome reporter carriers involving direct association with binding molecule.

1. Liposomes (preferably unilamellar vesicles with an average diameter of 150 to 300 nanometers) are prepared using the extrusion method (Hope, M. J., Bally, M. B., Webb, G., and Cullis, P. R., Biochimica et Biophysica Acta, 1985, 812:55-65); MacDonald, R. C., MacDonald, R. I., Menco, B., Takeshita, K., Subbarao, N., and Hu, L. Biochimica et Biophysica Acta, 1991, 1061:297-303). Other methods for liposome preparation may be used as well.

2. A solution of an oligopeptide, at a concentration 400 micromolar, is used during the preparation of the liposomes, such that the inner volume of the liposomes is loaded with this specific oligopeptide, which will serve to encode-decode the identity of a specific analyte of interest. A liposome with an internal diameter of 200 nanometers will contain, on the average, 960 molecules of the oligopeptide. Three separate preparations of liposomes are extruded, each loaded with a different oligopeptide. Short oligopeptides in the range of 1800 to 4000 daltons are chosen such that their respective masses will be different and readily separable by MALDI-TOF mass spectrometry.

3. The outer surface of the three liposome preparations is conjugated with specific antibodies, as follows: a) the first liposome preparation is reacted with an antibody specific for the p53 tumor suppressor; b) the second liposome preparation is reacted with an antibody specific for the Bcl-2 oncoprotein; c) the third liposome preparation is reacted with an antibody specific or the Her2/neu membrane receptor. Coupling reactions are performed using standard procedures for the covalent coupling of antibodies to molecules harboring reactive amino groups (Hendrickson, E. R., Hatfield, T. M., Joerger, R. D., Majarian, W. R., and Ebersole, R. C., 1995, Nucleic Acids Research, 23:522-529; Hermanson, G. T., Bioconjugate techniques, Academic Press, 1996, pp.528-569; Scheffold, A., Assenmacher, M., Reiners-Schramm, L., Lauster, R., and Radbruch, A., 2000, Nature Medicine 1:107-110). In the liposomes of this example, the reactive amino groups are those present in the phosphatidyl ethanolamine moieties of the liposomes.

4. A glass slide bearing a standard formaldehyde-fixed histological section is contacted with a mixture of all three liposome preparations, suspended in a buffer containing 30 mM Tris-HCl, pH 7.6, 100 mM Sodium Chloride, 1 mM EDTA, 0.1% Bovine serum albumin, in order to allow binding of the liposomes to the corresponding protein antigens present in the fixed tissue. After a one hour incubation, the slides are washed twice, for 5 minutes, with the same buffer (30 mM Tris-HCl, pH 7.6, 100 mM Sodium Chloride, 1 mM EDTA, 0.1% Bovine serum albumin). The slides are dried with a stream of air.

5. The slides are coated with a thin layer of matrix solution consisting of 10 mg/ml alpha-cyano-4-hydroxycinnamic acid, 0.1% trifluoroacetic acid in a 50:50 mixture of acetonitrile in water. The slides are dried with a stream of air.

6. The slide is placed on the surface of a modified MALDI plate, and introduced into a Voyager DE Pro instrument (PerSeptive PE Biosystems, Framingham, Mass.). The machine is run in positive-ion reflector mode, with an ion extraction delay time of 250 ns.

7. Mass spectra are obtained from defined positions on the slide surface. The relative amount of each of the three peaks of encoding polypeptides is used to decode the relative ratios of the Bcl-2, p53, and her2/neu antigens detected by the liposome reporter carriers.

Illustration 2:

The following illustrates use of examples of the disclosed liposome reporter carriers involving indirect association with binding molecule.

1. Liposomes (preferably unilamellar vesicles with an average diameter of 100 to 200 nanometers) are prepared using the extrusion method (Hope, M. J., Bally, M. B., Webb, G., and Cullis, P. R., Biochimica et Biophysica Acta, 1985, 812:55-65); MacDonald, R.C., MacDonald, R.I., Menco, B., Takeshita, K., Subbarao, N., and Hu, L. Biochimica et Biophysica Acta, 1991, 1061:297-303). Other methods for liposome preparation may be used as well.

2. A solution of an oligopeptide, at a concentration 400 micromolar, is used during the preparation of the liposomes, such that the inner volume of the liposomes is loaded with this specific oligopeptide, which will serve to encode- decode the identity of a specific analyte of interest. A liposome with an internal diameter of 200 nanometers will contain, on the average, 960 molecules of the oligopeptide. Three separate preparations of liposomes are extruded, each loaded with a different oligopeptide. Short oligopeptides in the range of 1800 to 4000 daltons are chosen such that their respective masses will be different and readily separable by MALDI-TOF mass spectrometry.

3. The outer surface of the three liposome preparations is conjugated with specific oligonucleotides, as follows: a) the first liposome preparation is covalently coupled with oligonucleotide SEQ ID NO:1; b) the second liposome preparation is covalently coupled with oligonucleotide SEQ ID NO:2; c) the third liposome preparation is covalently coupled with oligonucleotide SEQ ID NO:3. Coupling reactions are performed using standard procedures for the covalent coupling of oligonucleotides to molecules harboring reactive amino groups (Hendrickson, E. R., Hatfield, T. M., Joerger, R. D., Majarian, W. R., and Ebersole, R. C., 1995, Nucleic Acids Research, 23:522-529; Hernanson, G. T., Bioconjugate techniques, Academic Press, 1996, pp.528-569; Scheffold, A., Assenmacher, M., Reiners-Schramm, L., Lauster, R., and Radbruch, A., 2000, Nature Medicine 1:107-110). In the liposomes of this example, the reactive amino groups are those present in the phosphatidyl ethanolamine moieties.

4. Each of three specific antibodies is coupled to a specific DNA oligonucleotide, as follows: a) Oligonucleotide SEQ ID NO:4, which is complementary to Oligonucleotide SEQ ID NO:1, is reacted with an antibody specific for the p53 tumor suppressor; b) Oligonucleotide SEQ ID NO:5, which is complementary to Oligonucleotide SEQ ID NO:2, is reacted with an antibody specific for the Bcl-2 oncoprotein; c) Oligonucleotide SEQ ID NO:6, which is complementary to Oligonucleotide SEQ ID NO:3, is reacted with an antibody specific or the Her2/neu membrane receptor. Coupling reactions are performed using standard procedures for the covalent coupling of antibodies to oligonucleotides harboring reactive amino groups or sulfhydryl groups (Hendrickson, E. R., Hatfield, T. M., Joerger, R. D., Majarian, W. R., and Ebersole, R. C., 1995, Nucleic Acids Research, 23:522-529; Hermanson, G.T., Bioconjugate techniques, Academic Press, 1996, pp.528-569).

5. A glass slide bearing a standard formaldehyde-fixed histological section is contacted with a mixture containing all three antibody-DNA conjugates, suspended in a buffer containing 30 mM Tris-HCl, pH 7.6, 100 mM Sodium Chloride, 1 mM EDTA, 0.1% Bovine serum albumin, in order to allow binding of the antibody conjugates to the corresponding protein antigens present in the fixed tissue. After a 40 minute incubation, the slides are washed twice, for 5 minutes, with the same buffer (30 mM Tris-HCl, pH 7.6, 100 mM Sodium Chloride, 1 mM EDTA, 0.1% Bovine serum albumin).

6. The slide is then contacted with a mixture of all three liposome-DNA conjugates, suspended in a buffer containing 30 mM Tris-HCl, pH 7.6, 100 mM Sodium Chloride, 1 mM EDTA, 0.1% Bovine serum albumin, in order to allow binding of the liposome-DNA to the complementary DNA portion of the bound antibodies. After a one hour incubation, the slides are washed twice, for 5 minutes, with the same buffer (30 mM Tris-HCl, pH 7.6, 100 mM Sodium Chloride, 1 mM EDTA, 0.1% Bovine serum albumin). The slides are dried with a stream of air.

7. The slides are coated with a thin layer of matrix solution consisting of 10 mg/ml alpha-cyano-4-hydroxycinnamic acid, 0.1% trifluoroacetic acid in a 50:50 mixture of acetonitrile in water. The slides are dried with a stream of air.

8. The slides are placed on the surface of a modified MALDI plate, and introduced into a Voyager DE Pro instrument (PerSeptive PE Biosystems, Framingham, Mass.). The machine is run in positive-ion reflector mode, with an ion extraction delay time of 250 ns.

9. Mass spectra are obtained from defined positions on the slide surface. The relative amount of each of the three peaks of encoding polypeptides is used to decode the relative ratios of the Bcl-2, p53, and her2/neu antigens detected by the liposome-detector complexes.

The liposome carrier method is not limited to the detection of analytes on histological sections. Cells obtained by sorting may also be used for analysis according to this invention (Scheffold, A., Assenmacher, M., Reiners-Schramm, L., Lauster, R., and Radbruch, A., 2000, Nature Medicine 1:107-110).

Sequences

| | |
|---|---|
| 5′-GCATTCACGTCGATCGATTGCGAC-3′ | SEQ ID NO:1 |
| 5′-GCATGCAGTCGAGCGTAGCTAGGT-3′ | SEQ ID NO:2 |
| 5′-TAGGTACACTCAGCATGCAGCTAC-3′ | SEQ ID NO:3 |
| 5′-CGTAAGTGCAGCTAGCTAACGCTG-3′ | SEQ ID NO:4 |
| 5′-CGTACGTCAGCTCGCATCGATCCA-3′ | SEQ ID NO:5 |
| 5′-ATCCATGTGAGTCGTACGTCGATG-3′ | SEQ ID NO:6 |

Illustration 3:

The following illustrates use of examples of the disclosed bead reporter carriers using peptides as isobaric decoding tags.

1. Polystyrene beads of 0.2 micron diameter are derivatized according to Brummel et al., Science 264:399-402 (1994), to achieve the covalent binding of a single type of peptide (this is the decoding tag). The linkage chemistry incorporates a photocleavable bond (Olejnik et al., Proc Natl Acad Sci U S A 92:7590-7594 (1995)). Following this coupling reaction, the beads are derivatized further by coupling a specific anti-PSA (prostate specific antigen) monoclonal antibody (this is the specific binding molecule). After derivatization, each bead contains an average of 50,000 bound molecules of decoding tag, and 50 molecules of monoclonal antibody.

2. Using similar methods, another 9 bead preparations are modified in separate reactions, such that each bead class harbors a different decoding tag (belonging to a set of 10 different isobaric decoding tags), and a different monoclonal antibody. Thus, a single monoclonal antibody is associated with a single isobaric decoding tag.

3. A sample microarray is prepared, containing 196 microspots of dried serum, generated by spotting 0.2 microliters of human serum at different positions on the surface of a glass slide.

4. The 10 bead preparations are mixed in equal amounts, to obtain a preparation containing all classes of beads. The mixture is contacted with the serum microspot microarray in order to perform an immunoassay, where the 10 types of beads recognize 10 different antigens of interest present in the serum microspots. Each antigen of interest on a microspot is recognized by a specific antibody present in one of the bead classes. After incubation for 1 hour at 37 degrees, the excess unbound beads are washed away.

5. The microarray is coated with a suitable matrix for MALDI-TOF, introduced in a Quadrupole mass spectrometer, and a laser with a diameter of 100 microns is used to analize the isobaric decoding tags. Since the diameter of the laser is much larger than the average diameter of the particles, the laser generates a random, representative sampling of all the different beads bound of the microspot surface. The isobaric decoding tags present on the bound beads are released by photolysis of the photocleavable linkage. The spectrum of isobaric decoding tags indicates the spectrum of antigens present ion the serum microspot.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A method of detecting analytes, the method comprising associating one or more reporter carriers with one or more target samples, wherein the reporter carrier comprises a specific binding molecule, a carrier, and a plurality of decoding tags, and
detecting the decoding tags.

2. The method of claim 1, wherein the carrier is selected from the group consisting of liposomes, microparticles, nanoparticles, virons, phagmids, and branched polymer structures.

3. The method of claim 1, wherein the carrier is a liposome or microbead.

4. The method of claim 3, wherein the liposomes are unilamellar vesicles.

5. The method of claim 4, wherein the vesicles have an average diameter of 150 to 300 nanometers.

6. The method of claim 3, wherein the liposome has an internal diameter of 200 nanometers.

7. The method of claim 1, wherein the carrier is a dendrimer.

8. The method of claim 7, wherein the dendrimer is contacting a macromolecule selected from the group consisting of DNA, RNA, and PNA.

9. The method of claim 8, wherein the macromolecule is an oligonucleotide between 20 and 300 nucleotides in length.

10. The method of claim 1, wherein the specific binding molecule is selected from the group consisting of antibodies, ligands, binding proteins, receptor proteins, haptens, aptamers, carbohydrates, synthetic polyamides, and oligonucleotides.

11. The method of claim 1, wherein the specific binding molecule is a binding protein.

12. The method of claim 11, wherein the binding protein is a DNA binding protein.

13. The method of claim 11, wherein the DNA binding protein contains a motif selected from the group consisting of a zinc finger motif, leucine zipper motif, and helix-turn-helix motif.

14. The method of claim 1, wherein the specific binding molecule is an oligonucleotide.

15. The method of claim 14, wherein the oligonucleotide is between 10 and 40 nucleotides in length.

16. The method of claim 14, wherein the oligonucleotide is between 16 and 25 nucleotides in length.

17. The method of claim 14, wherein the oligonucleotide is a peptide nucleic acid.

18. The method of claim 14, wherein the oligonucleotide forms a triple helix with the target sequence.

19. The method of claim 14, wherein the oligonucleotide comprises a psoralen derivative capable of covalently attaching the oligonucleotide to the target sequence.

20. The method of claim 1, wherein the specific binding molecule is an antibody.

21. The method of claim 20, wherein the antibody binds a protein.

22. The method of claim 1, wherein the decoding tags are selected from the group consisting of oligonucleotides, carbohydrates, synthetic polyamides, peptide nucleic acids, antibodies, ligands, proteins, haptens, zinc fingers, aptamers, mass labels, and any combination of these.

23. The method of claim 1, wherein the decoding tags are peptide nucleic acids.

24. The method of claim 1, wherein the decoding tags are capable of hybridizing specifically to an oligonucleotide reporter tag.

25. The method of claim 24, wherein the length of the oligonucleotide reporter tag is between 10 and 35 nucleotides long.

26. The method of claim 24, wherein the length of the oligonucleotide reporter tag is between 15 and 20 nucleotides long.

27. The method of claim 1, wherein the decoding tags are capable of being detected by a method selected from the group consisting of nuclear magnetic resonance, electron paramagnetic resonance, surface enhanced raman scattering, surface plasmon resonance, fluorescence, phosphorescence, chemiluminescence, resonance raman, microwave, mass spectrometry, mass spectrometry electrophoresis chromatography, and any combination of these.

28. The method of claim 1, wherein the decoding tags are capable of being detected through MALDI-TOF spectroscopy.

29. The method of claim 1, wherein the specific binding molecule and the carrier are covalently linked.

30. The method of claim 1, wherein the carrier and the decoding tags are covalently linked.

31. The method of claim 30, wherein the specific binding molecule and the carrier are covalently linked.

32. The method of claim 1, wherein the specific binding molecule comprises a first oligonucleotide and the carrier comprises a second oligonucleotide which can hybridize to the first oligonucleotide.

33. The method of claim 32, wherein the first oligonucleotide is conjugated to an antibody which binds a protein.

34. The method of claim 1, wherein the decoding tags are isobaric decoding tags.

35. The method of claim 34, wherein a plurality of reporter carriers are associated with one or more target samples, wherein the decoding tags of each reporter carrier are different.

36. The method of claim 35, wherein the all of the decoding tags of all of the reporter carriers have the same mass-to-charge ratio.

37. The method of claim 36, wherein the decoding tags are altered by altering their mass, charge, or both, wherein the altered forms of the decoding tags are distinguished via differences in the mass-to-charge ratio of the altered forms of the decoding tags.

38. A composition for detecting an analyte comprising a specific binding molecule, a carrier, and a plurality of decoding tags.

39. The composition of claim 38, wherein the carrier is selected from the group consisting of liposomes, microparticles, nanoparticles, virons, phagmids, and branched polymer structures.

40. The composition of claim 38 wherein the carrier is a liposome.

41. The composition of claim 40, wherein the liposomes are unilamellar vesicles.

42. The composition of claim 41, wherein the vesicles have an average diameter of 150 to 300 nanometers.

43. The composition of claim 40, wherein the liposome has an internal diameter of 200 nanometers.

44. The composition of claim 38, wherein the carrier is a dendrimer.

45. The composition of claim 44, wherein the dendrimer is contacting a macromolecule selected from the group consisting of DNA, RNA, and PNA.

46. The composition of claim 45, wherein the macromolecule is an oligonucleotide between 20 and 300 nucleotides in length.

47. The composition of claim 38, wherein the specific binding molecule is selected from the group consisting of antibodies, ligands, binding proteins, receptor proteins, haptens, aptamers, carbohydrates, synthetic polyamides, and oligonucleotides.

48. The composition of claim 38, wherein the specific binding molecule is a binding protein.

49. The composition of claim 48, wherein the binding protein is a DNA binding protein.

50. The composition of claim 48, wherein the DNA binding protein contains a motif selected from the group consisting of a zinc finger motif, leucine zipper motif, and helix-turn-helix motif.

51. The composition of claim 38, wherein the specific binding molecule is an oligonucleotide.

52. The composition of claim 51, wherein the oligonucleotide is between 10 and 40 nucleotides in length.

53. The composition of claim 51, wherein the oligonucleotide is between 16 and 25 nucleotides in length.

54. The composition of claim 51, wherein the oligonucleotide is a peptide nucleic acid.

55. The composition of claim 51, wherein the oligonucleotide forms a triple helix with the target sequence.

56. The composition of claim 55, wherein the oligonucleotide comprises a psoralen derivative capable of covalently attaching the oligonucleotide to the target sequence.

57. The composition of claim 38, wherein the specific binding molecule is an antibody.

58. The composition of claim 57, wherein the antibody binds a protein.

59. The composition of claim 38, wherein the decoding tags are selected from the group consisting of oligonucleotides, carbohydrates, synthetic polyamides, peptide nucleic acids, antibodies, ligands, proteins, haptens, zinc fingers, aptamers, mass labels, and any combination of these.

60. The composition of claim 38, wherein the decoding tags are peptide nucleic acids.

61. The composition of claim 38, wherein the decoding tags are capable of hybridizing specifically to an oligonucleotide reporter tag.

62. The composition of claim 61, wherein the length of the oligonucleotide reporter tag is between 10 and 35 nucleotides long.

63. The composition of claim 61, wherein the length of the oligonucleotide reporter tag is between 15 and 20 nucleotides long.

64. The composition of claim 38, wherein the decoding tags are capable of being detected by a method selected from the group consisting of nuclear magnetic resonance, electron paramagnetic resonance, surface enhanced raman scattering, surface plasmon resonance, fluorescence, phosphorescence, chemiluminescence, resonance raman, microwave, mass spectrometry, mass spectrometry electrophoresis chromatography, and any combination of these.

65. The composition of claim 38, wherein the decoding tags are capable of being detected through MALDI-TOF spectroscopy.

66. The composition of claim 38, wherein the specific binding molecule and the carrier are covalently linked.

67. The composition of claim 38, wherein the carrier and the decoding tags are covalently linked.

68. The composition of claim 67, wherein the specific binding molecule and the carrier are covalently linked.

69. The composition of claim 38, wherein the specific binding molecule comprises a first oligonucleotide and the carrier comprises a second oligonucleotide which can hybridize to the first oligonucleotide.

70. The composition of claim 69, wherein the first oligonucleotide is conjugated to an antibody which binds a protein.

71. The composition of claim 38, wherein the decoding tags are isobaric decoding tags.


SEQUENCE LISTING (6 sequences)

SEQ ID NO: 1 - DNA, 24 bp, Artificial Sequence
  Description of artificial sequence = Synthetic construct
gcattcacgtcgatcgattgcgac

SEQ ID NO: 2 - DNA, 24 bp, Artificial Sequence
  Description of artificial sequence = Synthetic construct
gcatgcagtcgagcgtagctaggt

SEQ ID NO: 3 - DNA, 24 bp, Artificial Sequence
  Description of artificial sequence = Synthetic construct
taggtacactcagcatgcagctac

SEQ ID NO: 4 - DNA, 24 bp, Artificial Sequence
  Description of artificial sequence = Synthetic construct
cgtaagtgcagctagctaacgctg

SEQ ID NO: 5 - DNA, 24 bp, Artificial Sequence
  Description of artificial sequence = Synthetic construct
cgtacgtcagctcgcatcgatcca

SEQ ID NO: 6 - DNA, 24 bp, Artificial Sequence
  Description of artificial sequence = Synthetic construct
atccatgtgagtcgtacgtcgatg